(19) United States
(12) Patent Application Publication
(10) Pub. No.: US 2024/0034978 A1
(43) Pub. Date: Feb. 1, 2024

(54) Improved Airlift Bioreactor

(71) Applicant: The Better Meat Co., West Sacramento, CA (US)

(72) Inventor: Joshua Scott Rogers, Sacramento, CA (US)

(21) Appl. No.: 18/065,357

(22) Filed: Dec. 13, 2022

Related U.S. Application Data
(60) Provisional application No. 63/392,324, filed on Jul. 26, 2022.

(51) Int. Cl. C12M 1/00; C12M 1/12

(52) U.S. Cl. CPC C12M 29/08; C12M 27/20; C12M 29/18; C12M 23/06

(57) ABSTRACT

The present disclosure relates to a system including a main exterior body. The main exterior body includes (i) a return section at a bottom of the main exterior body, (ii) a gas disengagement section at a top of the main exterior body, (iii) a riser section positioned between the return section and the gas disengagement section, and (iv) a downcomer section positioned between the gas disengagement section and the return section. The system also includes one or more first spargers each having a first end and a second end opposite the first end. Each of the one or more first spargers are positioned such that the first end is outside of the main exterior body and the second end is positioned in a lower portion of the riser section of the main exterior body. The one or more first spargers are configured to supply compressed gas to the riser section of the main exterior body. The system also includes one or more second spargers each having a first end and a second end opposite the first end. Each of the one or more second spargers are positioned such that the first end is outside of the main exterior body and the second end is positioned in a lower portion of the downcomer section of the main exterior body adjacent to the return section. The one or more second spargers are configured to supply compressed gas to the downcomer section of the main exterior body.

### RELATED APPLICATIONS

This application claims priority to U.S. Provisional Application No. 63/392,324 entitled “Improved External Loop Airlift Bioreactor,” filed on Jul. 26, 2022, the contents of which are hereby incorporated by reference in its entirety.

### TECHNICAL FIELD

The present disclosure relates to a liquid circulation apparatus for continuously mixing a liquid, and a method of mixing such a liquid. More particularly, the invention provides an apparatus for use as an improved airlift bioreactor system.

### BACKGROUND

Unless otherwise indicated herein, the materials described in this section are not admitted to be prior art to the claims in this application.

An airlift bioreactor is a bioreactor in which the reaction medium is kept mixed and gassed by introduction of air or another gas (mixture) at the base of a column-like reactor equipped either with a draught tube or another device (e.g. external tube) by which the reactor volume is separated into a gassed and un-gassed region thus generating a vertically circulating flow. There are a variety of airlift bioreactors, including external-loop systems, internal-loop systems, split-cylinder systems, and bubble-column system, as non-limiting examples.

The external-loop airlift bioreactor (ELAB) is a specific type of fermenter that allows for the efficient aerobic fermentation of shear sensitive organisms that may or may not have high viscosities and/or be non-Newtonian in rheology. An ELAB is constructed of cylindrical sections combined to form a loop. It consists of three major areas of interest: the riser, the gas disengagement zone, and the downcomer. The riser and the downcomer are connected by the gas disengagement zone up top and the return section down below. In an ELAB, air is the driver of agitation and aeration, whereas in classical stir tank bioreactors air is the driver of aeration and an impeller is the driver of agitation. The fermentation liquid circulates in a loop in an ELAB.

The riser section of an ELAB is the section where air is introduced to function as a means for mass transfer and oxygen transfer in the fermentation. Because of this, the riser traditionally contains the highest volume of liquid in the bioreactor. The sparger system that delivers air in this section may be referred to as the main spargers. Liquid rises in the riser due to the injection of air from the main spargers, which causes lift through lowering the density of the fermentation liquid.

The gas disengagement zone section of an ELAB is the section where the air causing the lift in the riser disengages from the fermentation liquid. This section is at the top of the bioreactor and is specifically designed to facilitate the removal of oxygen and carbon dioxide from the fermentation.

The downcomer section of an ELAB is the section where the fermentation liquid falls. The fermentation liquid falls due to the absence of gas, which was removed from the liquid in the gas disengagement zone. This absence of gas (oxygen and carbon dioxide), causes the liquid density to increase, which causes the liquid to fall. Because of this, the downcomer section traditionally contains the lowest volume of liquid in the bioreactor. Sometimes one or more additional spargers are inserted near the top of the downcomer to introduce air that aerates the liquid at a rate not to exceed that to counter liquid circulation. This sparger is referred to as the one or more third spargers. The one or more third spargers may help prevent the fermentation from ever being truly devoid of oxygen.

In traditional ELABs, the bulk fluid may not be properly aerated when the liquid level drops below the top union point of an external loop airlift bioreactor (the part that contains the gas disengagement zone). Further, the downcomer is a zone that is typically free of aeration, meanwhile the fermentation is aerobic. ELABs traditionally have downcomers with smaller diameters than their Risers, which could lead to circulation blockages when fermenting viscous and/or filamentous organisms. In addition, ELABs traditionally have downcomers with smaller volumes than their risers in order to maximize the amount of aerated fluid which reduces the overall volume of the bioreactor. Accordingly, an improved airlift bioreactor to address the above disadvantages may be desirable.

### SUMMARY

In view of the foregoing, the present disclosure provides an improved airlift bioreactor system.

Thus, in a first aspect, the present disclosure provides a system including a main exterior body. The main exterior body includes (i) a return section at a bottom of the main exterior body, (ii) a gas disengagement section at a top of the main exterior body, (iii) a riser section positioned between the return section and the gas disengagement section, and (iv) a downcomer section positioned between the gas disengagement section and the return section. The system also includes one or more first spargers each having a first end and a second end opposite the first end. Each of the one or more first spargers are positioned such that the first end is outside of the main exterior body and the second end is positioned in a lower portion of the riser section of the main exterior body. The one or more first spargers are configured to supply compressed gas to the riser section of the main exterior body. The system also includes one or more second spargers each having a first end and a second end opposite the first end. Each of the one or more second spargers are positioned such that the first end is outside of the main exterior body and the second end is positioned in a lower portion of the downcomer section of the main exterior body adjacent to the return section. The one or more second spargers are configured to supply compressed gas to the downcomer section of the main exterior body.

These as well as other aspects, advantages, and alternatives, will become apparent to those of ordinary skill in the art by reading the following detailed description, with reference where appropriate to the accompanying drawings.

### BRIEF DESCRIPTION OF THE DRAWINGS

FIG. 1 illustrates a side view of an airlift bioreactor system, according to an exemplary embodiment.

FIG. 2 illustrates a top cross-sectional view of the one or more first spargers of the system of FIG. 1, according to an example embodiment.

FIG. 3 illustrates a top cross-sectional view of the one or more second spargers of the system of FIG. 1, according to an example embodiment

FIG. 4A illustrates a side cross-sectional view of the positioning of the one or more third spargers in the gas disengagement section, according to an example embodiment.

FIG. 4B illustrates a top cross-sectional view of the one or more third spargers of the system of FIG. 4A along line A-A, according to an example embodiment.

FIG. 4C illustrates a side cross-sectional view of the positioning of the one or more third spargers in the downcomer section, according to an example embodiment.

FIG. 4D illustrates a top cross-sectional view of the one or more third spargers of the system of FIG. 4B along line A-A, according to an example embodiment.

FIG. 5 illustrates a side view of an example sparger of the system of FIG. 1, according to an example embodiment.

FIG. 6A illustrates a side cross-sectional view of another airlift bioreactor system, according to an exemplary embodiment.

FIG. 6B illustrates a side cross-sectional view of another airlift bioreactor system, according to an exemplary embodiment.

FIG. 6C illustrates a side cross-sectional view of another airlift bioreactor system, according to an exemplary embodiment.

FIG. 6D illustrates a side cross-sectional view of another airlift bioreactor system, according to an exemplary embodiment.

### DETAILED DESCRIPTION

Example methods and systems are described herein. It should be understood that the words “example,” “exemplary,” and “illustrative” are used herein to mean “serving as an example, instance, or illustration.” Any embodiment or feature described herein as being an “example,” being “exemplary,” or being “illustrative” is not necessarily to be construed as preferred or advantageous over other embodiments or features. The exemplary embodiments described herein are not meant to be limiting. It will be readily understood that the aspects of the present disclosure, as generally described herein and illustrated in the figures, can be arranged, substituted, combined, separated, and designed in a wide variety of different configurations, all of which are explicitly contemplated herein.

Furthermore, the particular arrangements shown in the Figures should not be viewed as limiting. It should be understood that other embodiments may include more or less of each element shown in a given Figure. Further, some of the illustrated elements may be combined or omitted. Yet further, an exemplary embodiment may include elements that are not illustrated in the Figures.

As used herein, “coupled” means associated directly as well as indirectly. For example, a member A may be directly associated with a member B, or may be indirectly associated therewith, e.g., via another member C. It will be understood that not all relationships among the various disclosed elements are necessarily represented.

Unless otherwise indicated, the terms “first,” “second,” etc. are used herein merely as labels, and are not intended to impose ordinal, positional, or hierarchical requirements on the items to which these terms refer. Moreover, reference to, e.g., a “second” item does not require or preclude the existence of, e.g., a “first” or lower-numbered item, and/or, e.g., a “third” or higher-numbered item.

Reference herein to “one embodiment” or “one example” means that one or more feature, structure, or characteristic described in connection with the example is included in at least one implementation. The phrases “one embodiment” or “one example” in various places in the specification may or may not be referring to the same example.

As used herein, a system, apparatus, device, structure, article, element, component, or hardware “configured to” perform a specified function is indeed capable of performing the specified function without any alteration, rather than merely having potential to perform the specified function after further modification. In other words, the system, apparatus, structure, article, element, component, or hardware “configured to” perform a specified function is specifically selected, created, implemented, utilized, programmed, and/or designed for the purpose of performing the specified function. As used herein, “configured to” denotes existing characteristics of a system, apparatus, structure, article, element, component, or hardware which enable the system, apparatus, structure, article, element, component, or hardware to perform the specified function without further modification. For purposes of this disclosure, a system, apparatus, structure, article, element, component, or hardware described as being “configured to” perform a particular function may additionally or alternatively be described as being “adapted to” and/or as being “operative to” perform that function.

As used herein, with respect to measurements and angles, “about” means+/−5%.

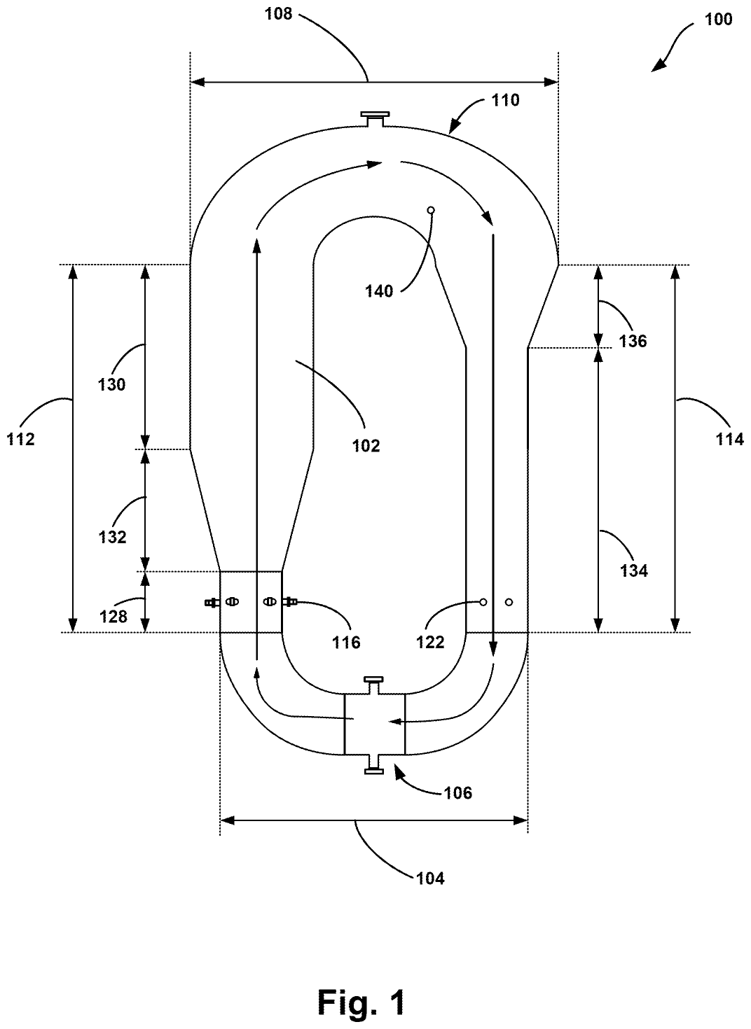

With reference to the Figures, FIG. 1 illustrates a system 100 including a main exterior body 102. The main exterior body 102 includes a return section 104 at a bottom 106 of the main exterior body 102, a gas disengagement section 108 at a top 110 of the main exterior body 102, a riser section 112 positioned between the return section 104 and the gas disengagement section 108, and a downcomer section 114 positioned between the gas disengagement section 108 and the return section 104. In one example, the riser section 112 is positioned between the return section 104 and the gas disengagement section 108 on a first side of the main exterior body 102, and the downcomer section 114 is positioned between the gas disengagement section 108 and the return section 104 on a second side of the main exterior body 102. The direction of fluid circulation is shown by the arrows in FIG. 1.

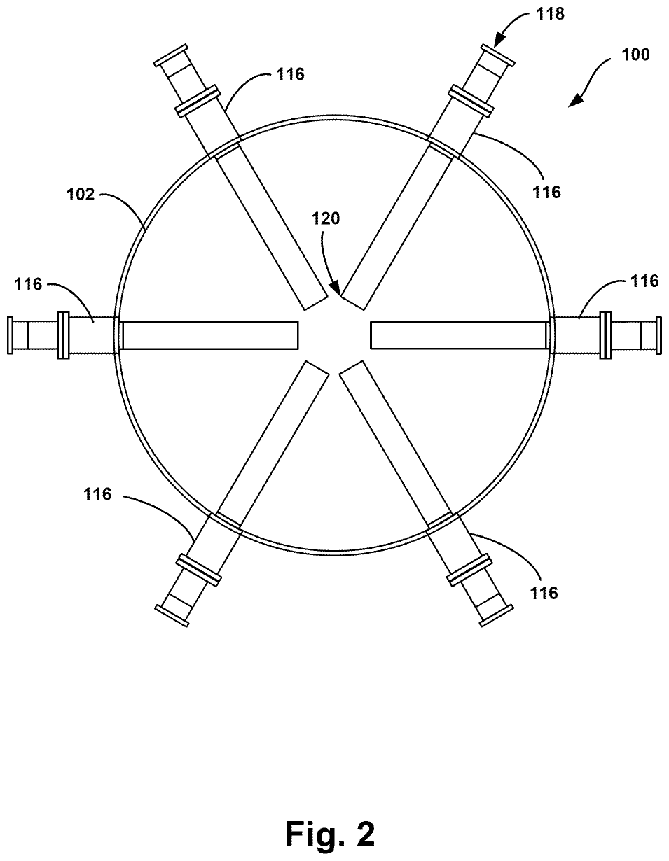

The system 100 further includes one or more first spargers 116 each having a first end 118 and a second end 120 opposite the first end. Each of the one or more first spargers 116 are positioned such that the first end 118 is outside of the main exterior body 102 and the second end 120 is positioned in an interior of the main exterior body 102 in a lower portion of the riser section 112, as shown in FIG. 2. The one or more first spargers 116 are configured to supply compressed gas to the riser section 112 of the main exterior body 102.

The one or more first spargers 116 may be configured to operate with a known amount of hydrostatic head and up to 20 psi of headspace pressure (typically between about 5 psi and about 14 psi), providing about 50 psig air delivery, and with a flow rate of up to about 1.5 VVM (typically between about 0.5 VVM to about 1.0 VVM). The one or more first spargers 116 are designed for aeration and agitation. In one example, the one or more first spargers 116 operate at ˜90% of total reactor sparge during reaction. The fermentation reaction itself forms a fluid that becomes very viscous near its end. In some embodiments, the one or more first spargers 116 may be configured as removable tri-clamp spargers for cleaning out of place (COP) and which can be arranged in a form of an array, as discussed in additional detail below. In one example, each of the one or more first spargers 116 comprise 1.5″ diameter tubes with a porous section having a length of about 9.5″.

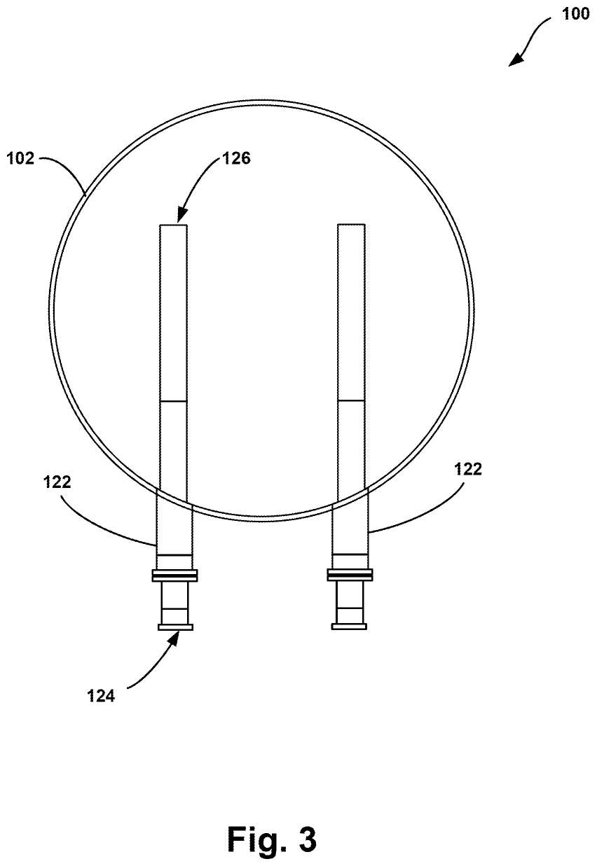

The system 100 further includes one or more second spargers 122 each having a first end 124 and a second end 126 opposite the first end 124. Each of the one or more second spargers 122 are positioned such that the first end 124 is outside of the main exterior body 102 and the second end 126 is positioned in an interior of the main exterior body 102 in a lower portion of the downcomer section 114 adjacent to the return section 104, as shown in FIG. 3. The one or more second spargers 122 are configured to supply compressed gas to the downcomer section 114 of the main exterior body 102. As used herein, the compressed gas may comprise air, oxygen, and/or other gases that can be used to drive agitation and/or aeration within the system 100.

The one or more second spargers 122 may be configured to operate with a known amount of hydrostatic head and up to 20 psi of headspace pressure (typically between about 5 psi and about 14 psi), providing about 50 psig air delivery, and with a flow rate of up to about 0.5 VVM (typically between about 0.1 VVM to about 0.25 VVM). The one or more second spargers 122 are designed to be used when downcomer section 114 is isolated for aeration and agitation (e.g., in a bubble column configuration). As described above, the fermentation reaction itself forms a fluid that becomes very viscous near its end. In some embodiments, the one or more second spargers 122 may be configured as removable tri-clamp spargers for cleaning out of place (COP) and which can be arranged in a form of an array, as discussed in additional detail below. In one example, each of the one or more second spargers 122 comprise 1.5″ diameter tubes with a porous section having a length of about 10″.

The presence of the one or more second spargers 122 provides a variety of benefits over a traditional airlift bioreactor systems. In particular, the one or more second spargers 122 allow the system 100 to be operated as dual bubble columns when the system 100 volume is too low to circulate, such as during a drain-and-fill operation. This maintains aerobic conditions in the downcomer section 114 in order to prevent sub-optimal growth conditions and, by extension, detrimental physiological changes of the organism. Further, the one or more second spargers 122 allow the system 100 to be operated in fed-batch mode, where the initial liquid volume of the batch is too low for circulation, but is sufficient for circulation after the feed is added. Further, the one or more second spargers 122 allow extra aeration in the downcomer section 114 when a fermentation is running in recirculation mode, allow the circulation pattern to be run in reverse, to dislodge accumulations of biomass in the system 100, and allow the downcomer section 114 to have a larger diameter and volume, while maintaining aeration, to increase general capacity of the system 100, and allow the system 100 to be aerated as two bubble columns if the volume drops below the top union point due to foam-out or evaporation.

In one example, as shown in FIG. 1, the system 100 comprises an external-loop system, and wherein the main exterior body comprises a closed loop. In one such example, each of the return section 104, the gas disengagement section 108, the riser section 112, and the downcomer section 114 have a round cross-section.

In one example, a diameter of the return section 104 is less than a diameter of the gas disengagement section 108. In another example, the riser section 112 includes a first portion 128 having a first diameter, a second portion 130 having a second diameter that is greater than the first diameter, and a third portion 132 having a variable diameter connecting the first portion to the second portion. In one such example, the second portion 130 of the riser section 112 has a greater length than the first portion 128 of the riser section 112. In another example, the first diameter of the first portion 128 of the riser section 112 is equal to a diameter of the return section 104. In another example, the second diameter of the second portion 130 of the riser section 112 is equal to a diameter of the gas disengagement section 108. In one example, the one or more first spargers 116 are positioned in the first portion 128 of the riser section 112.

In another example, the downcomer section 114 includes a first portion 134 having a first diameter, and a second portion 136 having a variable diameter connecting the first portion 134 to the gas disengagement section 108. In one such example, the first portion 134 of the downcomer section 114 has a greater length than the second portion 136 of the downcomer section 114. In another example, the first diameter of the first portion 134 of the downcomer section 114 is equal to a diameter of the return section 104. In another example, the second diameter of the second portion 136 of the downcomer section 114 is equal to a diameter of the gas disengagement section 108. In one example, the one or more second spargers 122 are positioned in the first portion 134 of the downcomer section 114.

In one example, as shown in FIG. 2, the one or more first spargers 116 may comprise a plurality of spargers equally spaced around the riser section 112 such that a longitudinal axis of each of the one or more first spargers 116 intersect at a center of the riser section. In one particular example, the one or more first spargers 116 comprises six spargers. In one example, as shown in FIG. 2, each of the one or more first spargers 116 are positioned such that the first end 118 is outside of the main exterior body 102.

In one example, as shown in FIG. 3, the one or more second spargers 122 comprises two spargers. In one example, the one or more second spargers 122 are positioned such that a longitudinal axis of each of the one or more second spargers 122 are parallel to one another. In one example, as shown in FIG. 3, each of the one or more second spargers are positioned such that the first end 124 is outside of the main exterior body 102.

The system 100 described herein is configured to be operated such that a circular liquid motion is induced by compressed gas from the one or more first spargers 116 and/or the one or more second spargers 122. The system 100 described herein is configured to be operated such that a circular liquid motion is induced by gas from the one or more first spargers 116 and/or the one or more second spargers 122. In one example, a pressure of the compressed gas supplied by the one or more first spargers 116 to the riser section 112 of the main exterior body 102 is adjustable, and a pressure of the compressed gas supplied by the one or more second spargers 122 to the downcomer section 114 of the main exterior body 102 is adjustable. In one example, the pressure of the compressed gas supplied by the one or more first spargers 116 to the riser section 112 of the main exterior body 102 is greater than the pressure of the compressed gas supplied by the one or more second spargers 122 to the downcomer section 114 of the main exterior body 102. In another example, a pressure of the compressed gas supplied by the one or more first spargers 116 to the riser section 112 of the main exterior body 102 is less than a pressure of the compressed gas supplied by the one or more second spargers 122 to the downcomer section 114 of the main exterior body 102. In another example, flow through the one or more first spargers 116 and/or the one or more second spargers 122 can be shut off or otherwise deactivated to thereby adjust the pressure differential in the system 100. In one example, the system 100 further includes one or more impellers to aid in mixing.

By adjusting the pressure of the compressed gas supplied to the one or more first spargers 116 and/or the one or more second spargers 122, it is possible to reverse the direction of the circulation of the fermentation medium or fluid which helps dislodge accumulations of biomass in the system 100 while also adding additional oxygen to the media.

In one example, as shown in FIG. 1, the system 100 includes one or more third spargers 140 each having a first end 142 and a second end 144 opposite the first end 142. In one example, each of the one or more third spargers 140 are positioned such that the first end 142 is outside of the main exterior body 102 and the second end 144 is positioned in an interior of the main exterior body 102 in the gas disengagement section 108. In such an example, the one or more third spargers 140 are configured to supply compressed gas to the gas disengagement section 108 of the main exterior body 102. In another example, each of the one or more third spargers 140 are positioned such that the first end 142 is outside of the main exterior body 102 and the second end 144 is positioned in an interior of the main exterior body 102 in the second portion 136 of the downcomer section 114. In such an example, the one or more third spargers 140 are configured to supply compressed gas to second portion 136 of the downcomer section 114 of the main exterior body 102.

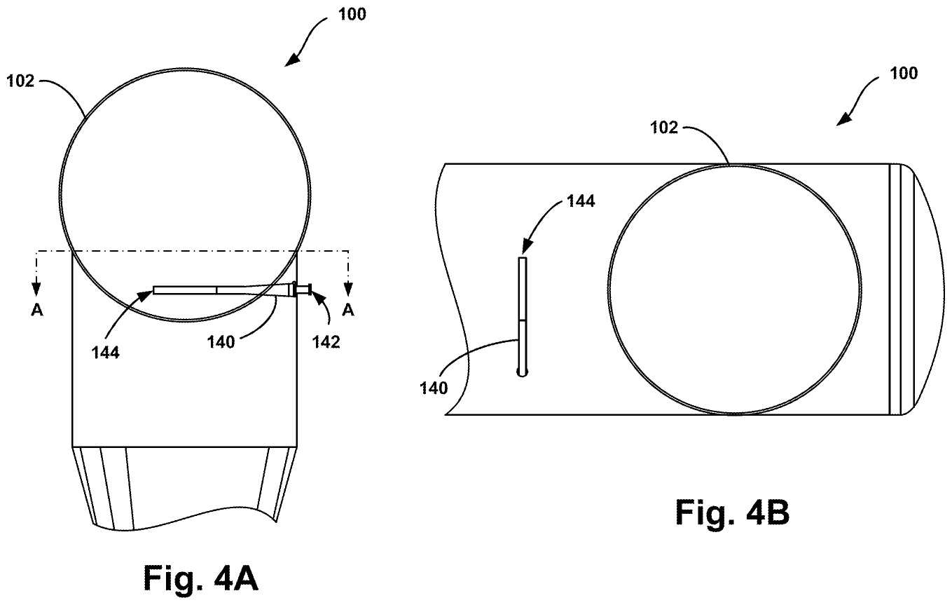

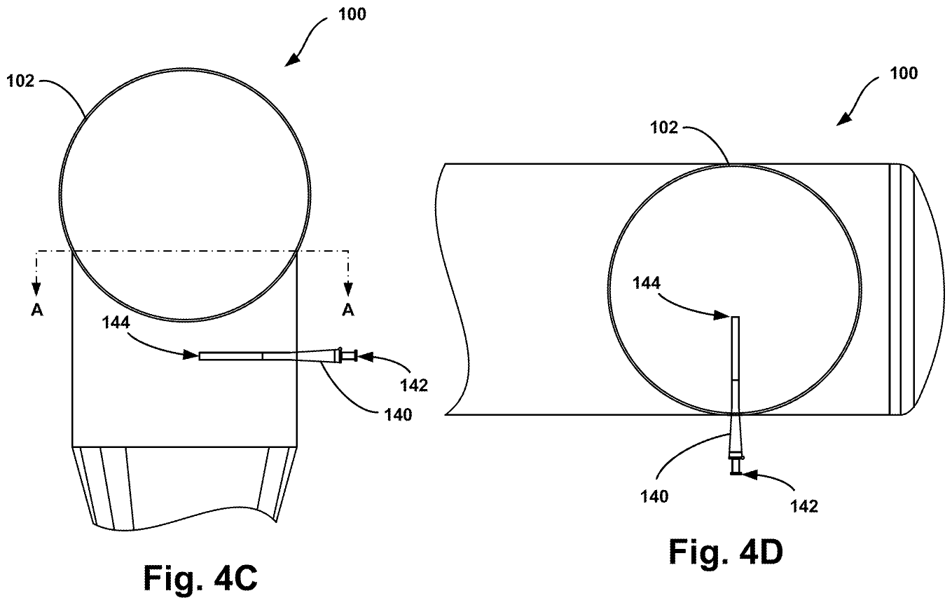

In one example, as shown in FIGS. 1 and 4A-4D, the one or more third spargers 140 comprise a single sparger. In one such example, as shown in FIG. 1, the single sparger of the one or more third spargers 140 is positioned closer to the downcomer section 114 than the riser section 112. In another example, as shown in FIGS. 4A-4B, the single sparger of the one or more third spargers 140 is positioned on a bottom half of the gas disengagement section 108. In yet another example, as shown in FIGS. 4C-4D, the single sparger of the one or more third spargers 140 is positioned in the second portion 136 of the downcomer section 114 of the main exterior body 102.

The one or more third spargers 140 may be configured to operate with a known amount of hydrostatic head and up to 20 psi of headspace pressure (typically between about 5 psi and about 14 psi), providing about 50 psig air delivery, and with a flow rate of up to about 0.15 VVM (typically between about 0.05 VVM to about 0.1 VVM). The one or more third spargers 140 are designed for aeration only. In one example, the one or more third spargers 140 operate at ˜10% of total reactor sparge during reaction. As described above, the fermentation reaction itself forms a fluid that becomes very viscous near its end. In some embodiments, the one or more third spargers 140 may be configured as removable tri-clamp spargers for cleaning out of place (COP) and which can be arranged in a form of an array, as discussed in additional detail below. In one example, each of the one or more third spargers 140 comprise 1.5″ diameter tubes with a porous section having a length of about 12″.

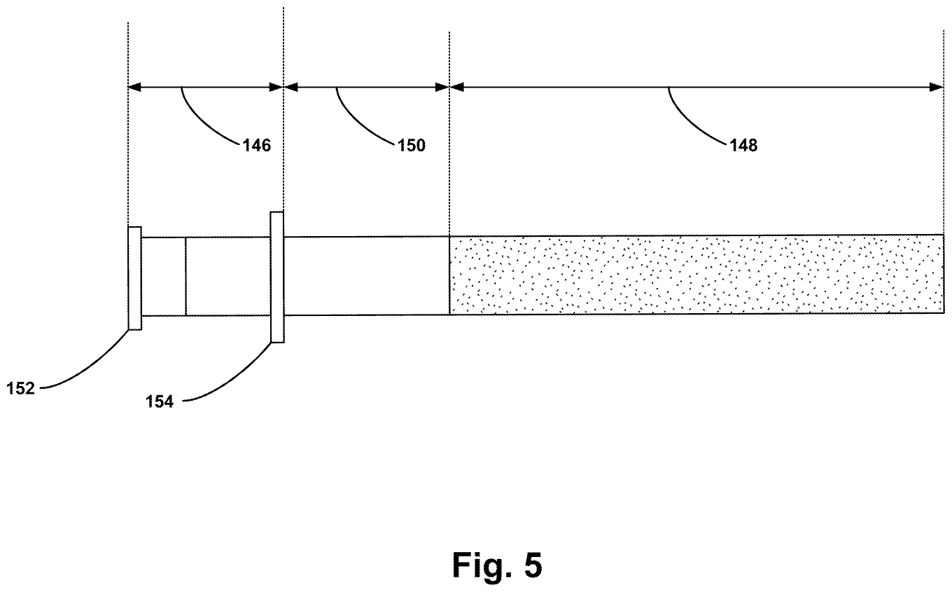

FIG. 5 illustrates an example sparger of the system 100. The sparger shown in FIG. 5 may be one of the one or more first spargers 116, one of the one or more second spargers 122, or one of the one or more third spargers 140. As shown in FIG. 5, each of the spargers may include a first portion 146 of the spargers resides outside of the system 100, a porous section 148 that resides inside of the system 100 at the second end for air distribution, and a second section 150 that resides inside of the system 100 and is positioned between the first portion 146 and the porous section 148. In use, compressed air enters the spargers through a first tri-clamp connection 152 and injects into the system 100 through the porous section 148. In one example, the spargers are removable and are connected to the system 100 a second tri-clamp connection 154. In one example, the first tri-clamp connection 152 comprises a 1.5″ connection, and the second tri-clamp connection 154 comprises a 2″ connection. In one example, a length of the porous section of the one or more first spargers 116 is less than a length of the porous section of the one or more second spargers 122, which is less than a length of the porous section of the one or more third spargers 140. In another example, a length of the porous of the one or more first spargers 116, a length of the porous section of the one or more second spargers 122, and a length of the porous section of the one or more third spargers 140 are equal.

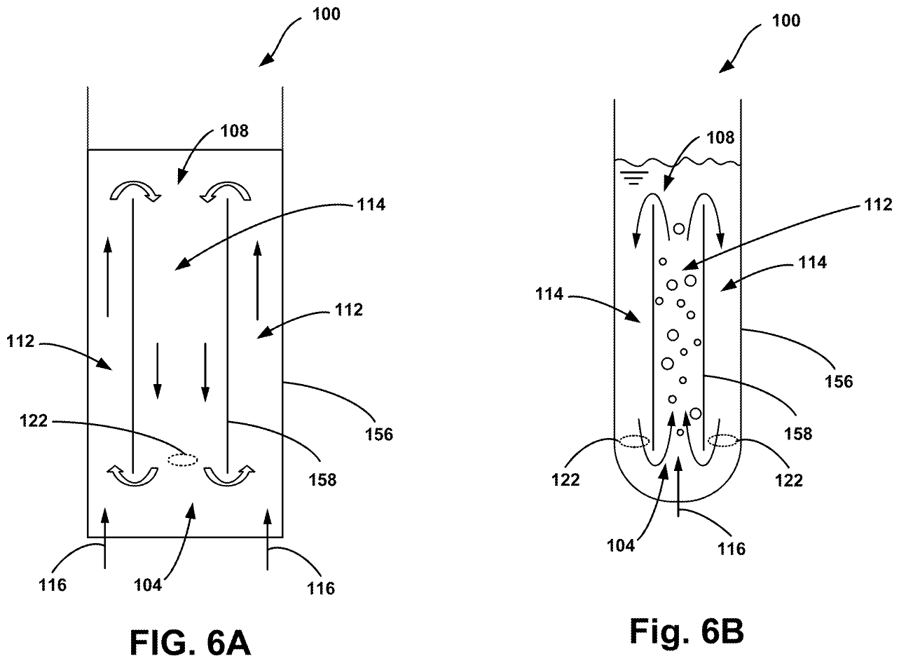

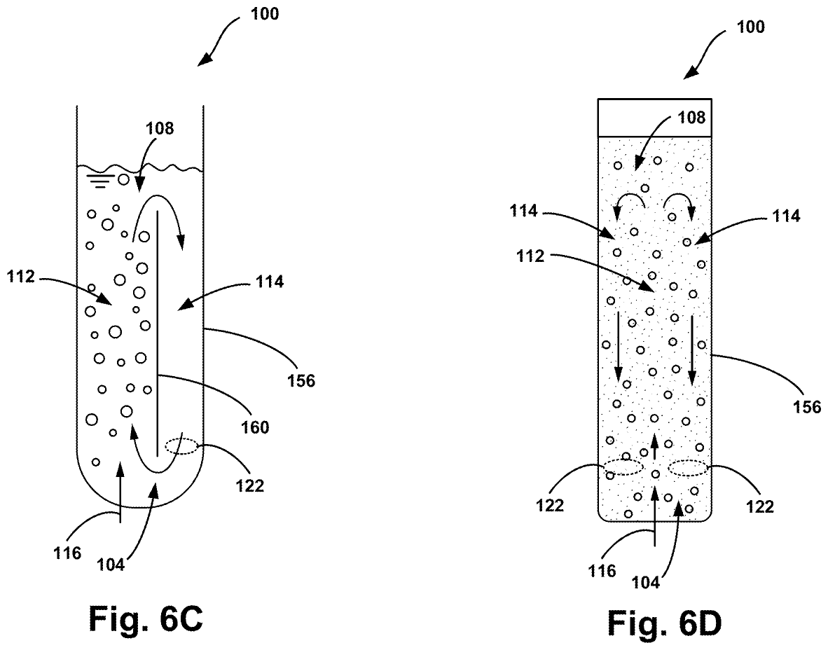

Although FIG. 1 illustrates an external-loop system, other airlift bioreactors may also benefit from the arrangements described above by including the one or more second spargers 122. In particular, as shown in FIGS. 6A-6B, in one example the system 100 comprises a draft-tube internal-loop system, where the main exterior body 102 comprises a cylinder 156 with an inner tube 158 positioned therein. In one such example, as shown in FIG. 6A, the riser section is positioned on either side of the inner tube 158. In such an example, the one or more first spargers 116 are positioned at the bottom of the riser sections 112 on either side of the inner tube 158. Further, the one or more second spargers 122 are positioned in a lower portion of the downcomer section 114 adjacent to the return section 104 in line with a bottom of the inner tube 158.

In another example, as shown in FIG. 6B, the riser section is positioned in the inner tube 158. In such an example, the one or more first spargers 116 are positioned at the bottom of the riser section 112 in line with a bottom of the inner tube 158. Further, the one or more second spargers 122 are positioned in a lower portion of the downcomer sections 114 adjacent to the return section 104 on either side of the inner tube 158. As described above, by adjusting the pressure of the compressed gas supplied to the one or more first spargers 116 and/or the one or more second spargers 122, it is possible to reverse the direction of the circulation of the fermentation medium or fluid which helps dislodge accumulations of biomass in the system 100 while also adding additional oxygen to the media. Such an example is shown in the varying directions of flown seen in FIGS. 6A-6B.

In another example, as shown in FIG. 6C, the system 100 comprises a split-cylinder system, where the main exterior body 102 comprises a cylinder 156 with a wall or a baffle 160 positioned therein. In such an example, the one or more first spargers 116 are positioned at the bottom of the riser section 112 on one side of the wall or baffle 160. Further, the one or more second spargers 122 are positioned in a lower portion of the downcomer section 114 adjacent to the return section 104. The one or more second spargers 122 are positioned on an opposite side of the wall or baffle 160 from the one or more first spargers 116.

In yet another example, the system 100 comprises a bubble-column system, where the main exterior body comprises a cylinder 156. In such an example, the one or more first spargers 116 are positioned at the bottom of the riser section 112 in a center of the system 100. Further, the one or more second spargers 122 are positioned in a lower portion of the downcomer section 114 adjacent to the return section 104 on either side of the one or more first spargers 116.

### Example 1—Fermentation Process Using the Improved Airlift Bioreactor

A filamentous organism Neurospora crassa was grown in a submerged manner utilizing the system 100 described above. The end product is the dried biomass harvested from this fermentation, which is high in nutritional content and used as a food ingredient.

The process starts with a seed vial that contains a known amount of cells. A 2 L baffled Erlenmeyer seed flask is filled with nutrient rich liquid media composed of potato dextrose. This flask is heat sterilized and then inoculated with a known amount of cells (10×10\hat{ }6 conidia/mL) from the seed vial. This inoculated flask is incubated on a shaker table at a set speed of about 100 to 200 RPM and temperature of about 30 to 35 Celsius for about 24 to 48 hours.

A 200 L agitated seed tank is filled with nutrient rich liquid media. This tank is heat sterilized and then inoculated with about 2 L of culture. This inoculated seed tank is set to a known air flow of about 0.5 to 1.2 VVM, temperature of 27 to 35 Celsius, pressure of about 5 to 15 psig, stir speed of about 200 to 600 RPM and allowed to ferment for a set amount of time of about 24 to 48 hours.

The system 100 is filled with pre-sterilized nutrient rich liquid media from any one of the top nozzles as shown in FIG. 1. The bioreactor is then inoculated with a known volume of about 200 L of culture from the previously mentioned seed tank. This inoculated bioreactor is set to a known air flow of about 0.5 to 1.2 VVM, temperature of about 27 to 35 Celsius, pressure of about 5 to 15 psig and allowed to ferment for about 24 to 48 hours depending on growth and nutrient consumption. The air flow is introduced at various locations in the bioreactor depending on what is needed for the fermentation to progress. These locations include the one or more first spargers 116 for aeration and agitation, the one or more third spargers 140 for supplemental aeration, and/or the one or more second spargers 122 for improved operation as highlighted above. The specific locations of these spargers in the bioreactor can be seen FIG. 1 above.

Once the fermentation in system 100 is complete, the biomass filled liquid, also known as slurry, is pressure transferred out of the bottom nozzle of the system 100 to a downstream processing area where water is removed from the slurry through the use of mechanical and thermal means. The resulting dry product is sized and packaged to be used as a shelf stable ingredient for food. See, for instance, U.S. Pat. No. 11,058,137, which is incorporated by reference in its entirety.

Without limiting the present disclosure, a number of embodiments of the present disclosure are described below for purpose of illustration.

Embodiment 1: A system comprising:
a main exterior body comprising:
   a return section at a bottom of the main exterior body;
   a gas disengagement section at a top of the main exterior body;
   a riser section positioned between the return section and the gas disengagement section; and
   a downcomer section positioned between the gas disengagement section and the return section;
one or more first spargers each having a first end and a second end opposite the first end, wherein each of the one or more first spargers are positioned such that the first end is outside of the main exterior body and the second end is positioned in a lower portion of the riser section of the main exterior body, and wherein the one or more first spargers are configured to supply compressed gas to the riser section of the main exterior body; and
one or more second spargers each having a first end and a second end opposite the first end, wherein each of the one or more second spargers are positioned such that the first end is outside of the main exterior body and the second end is positioned in a lower portion of the downcomer section of the main exterior body adjacent to the return section, and wherein the one or more second spargers are configured to supply compressed gas to the downcomer section of the main exterior body.

Embodiment 2: The system of embodiment 1, wherein the system comprises an external-loop system, and wherein the main exterior body comprises a closed loop.

Embodiment 3: The system of embodiment 2, wherein each of the return section, the gas disengagement section, the riser section, and the downcomer section have a round cross-section.

Embodiment 4: The system of any one of embodiments 2-3, wherein a diameter of the return section is less than a diameter of the gas disengagement section.

Embodiment 5: The system of any one of embodiments 2-4, wherein the riser section includes a first portion having a first diameter, a second portion having a second diameter that is greater than the first diameter, and a third portion having a variable diameter connecting the first portion to the second portion.

Embodiment 6: The system of embodiment 5, wherein the second portion of the riser section has a greater length than the first portion of the riser section.

Embodiment 7: The system of any one of embodiments 5-6, wherein the first diameter of the first portion of the riser section is equal to a diameter of the return section.

Embodiment 8: The system of any one of embodiments 5-7, wherein the second diameter of the second portion of the riser section is equal to a diameter of the gas disengagement section.

Embodiment 9: The system of any one of embodiments 5-8, wherein the one or more first spargers are positioned in the first portion of the riser section.

Embodiment 10: The system of any one of embodiments 2-9, wherein the downcomer section includes a first portion having a first diameter and a second portion having a variable diameter connecting the first portion to the gas disengagement section.

Embodiment 11: The system of embodiment 10, wherein the first portion of the downcomer section has a greater length than the second portion of the downcomer section.

Embodiment 12: The system of any one of embodiments 10-11, wherein the first diameter of the first portion of the downcomer section is equal to a diameter of the return section.

Embodiment 13: The system of any one of embodiments 10-12, wherein the one or more second spargers are positioned in the first portion of the downcomer section.

Embodiment 14: The system of any one of embodiments 2-13, wherein the one or more first spargers comprises a plurality of spargers equally spaced around the riser section such that a longitudinal axis of each of the one or more first spargers intersect at a center of the riser section.

Embodiment 15: The system of embodiment 14, wherein the one or more first spargers comprises six spargers.

Embodiment 16: The system of any one of embodiments 1-15, wherein the one or more second spargers comprises two spargers.

Embodiment 17: The system of any one of embodiments 1-16, wherein the one or more second spargers are positioned such that a longitudinal axis of each of the one or more second spargers are parallel to one another.

Embodiment 18: The system of any one of embodiments 1-17, further comprising:
one or more third spargers each having a first end and a second end opposite the first end, wherein each of the one or more third spargers are positioned such that the first end is outside of the main exterior body and the second end is positioned in one of (i) the gas disengagement section of the main exterior body or (ii) a second portion of the downcomer section of the main exterior body.

Embodiment 19: The system of embodiment 18, wherein the one or more third spargers comprise a single sparger.

Embodiment 20: The system of embodiment 19, wherein the single sparger of the one or more third spargers is positioned closer to the downcomer section than the riser section.

Embodiment 21: The system of embodiment 20, wherein the single sparger of the one or more third spargers is positioned on a bottom half of the gas disengagement section.

Embodiment 22: The system of any one of embodiments 1-21, wherein each of the one or more first spargers include a porous section at the second end, and wherein each of the one or more second spargers include a porous section at the second end.

Embodiment 23: The system of any one of embodiments 1-22, wherein the riser section is positioned between the return section and the gas disengagement section on a first side of the main exterior body, and wherein the downcomer section is positioned between the gas disengagement section and the return section on a second side of the main exterior body.

Embodiment 24: The system of any one of embodiments 1-23, wherein each of the one or more first spargers are positioned such that the first end is outside of the main exterior body, and wherein each of the one or more second spargers are positioned such that the first end is outside of the main exterior body.

Embodiment 25: The system of any one of embodiments 1-24, wherein a pressure of the compressed gas supplied by the one or more first spargers to the riser section of the main exterior body is adjustable, and wherein a pressure of the compressed gas supplied by the one or more second spargers to the downcomer section of the main exterior body is adjustable.

Embodiment 26: The system of any one of embodiments 1-25, wherein a pressure of the compressed gas supplied by the one or more first spargers to the riser section of the main exterior body is greater than a pressure of the compressed gas supplied by the one or more second spargers to the downcomer section of the main exterior body.

Embodiment 27: The system of any one of embodiments 1-26, wherein a pressure of the compressed gas supplied by the one or more first spargers to the riser section of the main exterior body is less than a pressure of the compressed gas supplied by the one or more second spargers to the downcomer section of the main exterior body.

Embodiment 28: The system of any one of embodiments 1-27, wherein the system comprises a draft-tube internal-loop system, wherein the main exterior body comprises a cylinder with an inner tube positioned therein.

Embodiment 29: The system of embodiment 28, wherein the riser section is positioned in the inner tube.

Embodiment 30: The system of embodiment 28, wherein the riser section is positioned on either side of the inner tube.

Embodiment 31: The system of any one of embodiments 1-30, wherein the system comprises a split-cylinder system, and wherein the main exterior body comprises a cylinder with a wall or a baffle positioned therein.

Embodiment 32: The system of any one of embodiments 1-31, wherein the system comprises a bubble-column system, and wherein the main exterior body comprises a cylinder.

Embodiment 33: The system of any one of embodiments 1-32, wherein the system is configured to be operated such that a circular liquid motion is induced by gas from the one or more first spargers and/or the one or more second spargers.

Embodiment 34: The system of any one of embodiments 1-33, wherein the system includes one or more impellers to aid in mixing.

While various aspects and embodiments have been disclosed herein, other aspects and embodiments will be apparent to those skilled in the art. The various aspects and embodiments disclosed herein are for purposes of illustration and are not intended to be limiting, with the true scope being indicated by the following claims, along with the full scope of equivalents to which such claims are entitled. It is also to be understood that the terminology used herein is for the purpose of describing particular embodiments only, and is not intended to be limiting.

Because many modifications, variations, and changes in detail can be made to the described example, it is intended that all matters in the preceding description and shown in the accompanying figures be interpreted as illustrative and not in a limiting sense. Further, it is intended to be understood that the following clauses (and any combination of the clauses) further describe aspects of the present description.

1. A system comprising:
a main exterior body comprising:
a return section at a bottom of the main exterior body;
a gas disengagement section at a top of the main exterior body;
a riser section positioned between the return section and the gas disengagement section; and
a downcomer section positioned between the gas disengagement section and the return section;
one or more first spargers each having a first end and a second end opposite the first end, wherein each of the one or more first spargers are positioned such that the first end is outside of the main exterior body and the second end is positioned in a lower portion of the riser section of the main exterior body, and wherein the one or more first spargers are configured to supply compressed gas to the riser section of the main exterior body; and
one or more second spargers each having a first end and a second end opposite the first end, wherein each of the one or more second spargers are positioned such that the first end is outside of the main exterior body and the second end is positioned in a lower portion of the downcomer section of the main exterior body adjacent to the return section, and wherein the one or more second spargers are configured to supply compressed gas to the downcomer section of the main exterior body.

2. The system of claim 1, wherein the system comprises an external-loop system, and wherein the main exterior body comprises a closed loop.

3. The system of claim 2, wherein each of the return section, the gas disengagement section, the riser section, and the downcomer section have a round cross-section.

4. The system of claim 2, wherein a diameter of the return section is less than a diameter of the gas disengagement section.

5. The system of claim 2, wherein the riser section includes a first portion having a first diameter, a second portion having a second diameter that is greater than the first diameter, and a third portion having a variable diameter connecting the first portion to the second portion.

6. The system of claim 5, wherein the second portion of the riser section has a greater length than the first portion of the riser section.

7. The system of claim 5, wherein the first diameter of the first portion of the riser section is equal to a diameter of the return section.

8. The system of claim 5, wherein the second diameter of the second portion of the riser section is equal to a diameter of the gas disengagement section.

9. The system of claim 5, wherein the one or more first spargers are positioned in the first portion of the riser section.

10. The system of claim 2, wherein the downcomer section includes a first portion having a first diameter and a second portion having a variable diameter connecting the first portion to the gas disengagement section.

11. The system of claim 10, wherein the first portion of the downcomer section has a greater length than the second portion of the downcomer section.

12. The system of claim 10, wherein the first diameter of the first portion of the downcomer section is equal to a diameter of the return section.

13. The system of claim 10, wherein the one or more second spargers are positioned in the first portion of the downcomer section.

14. The system of claim 2, wherein the one or more first spargers comprises a plurality of spargers equally spaced around the riser section such that a longitudinal axis of each of the one or more first spargers intersect at a center of the riser section.

15. The system of claim 14, wherein the one or more first spargers comprises six spargers.

16. The system of claim 1, wherein the one or more second spargers comprises two spargers.

17. The system of claim 1, wherein the one or more second spargers are positioned such that a longitudinal axis of each of the one or more second spargers are parallel to one another.

18. The system of claim 1, further comprising:
one or more third spargers each having a first end and a second end opposite the first end, wherein each of the one or more third spargers are positioned such that the first end is outside of the main exterior body and the second end is positioned in one of (i) the gas disengagement section of the main exterior body or (ii) a second portion of the downcomer section of the main exterior body.

19. The system of claim 18, wherein the one or more third spargers comprise a single sparger.

20. The system of claim 19, wherein the single sparger of the one or more third spargers is positioned closer to the downcomer section than the riser section.

21. The system of claim 20, wherein the single sparger of the one or more third spargers is positioned on a bottom half of the gas disengagement section.

22. The system of claim 1, wherein each of the one or more first spargers include a porous section at the second end, and wherein each of the one or more second spargers include a porous section at the second end.

23. The system of claim 1, wherein the riser section is positioned between the return section and the gas disengagement section on a first side of the main exterior body, and wherein the downcomer section is positioned between the gas disengagement section and the return section on a second side of the main exterior body.

24. The system of claim 1, wherein each of the one or more first spargers are positioned such that the first end is outside of the main exterior body, and wherein each of the one or more second spargers are positioned such that the first end is outside of the main exterior body.

25. The system of claim 1, wherein a pressure of the compressed gas supplied by the one or more first spargers to the riser section of the main exterior body is adjustable, and wherein a pressure of the compressed gas supplied by the one or more second spargers to the downcomer section of the main exterior body is adjustable.

26. The system of claim 1, wherein a pressure of the compressed gas supplied by the one or more first spargers to the riser section of the main exterior body is greater than a pressure of the compressed gas supplied by the one or more second spargers to the downcomer section of the main exterior body.

27. The system of claim 1, wherein a pressure of the compressed gas supplied by the one or more first spargers to the riser section of the main exterior body is less than a pressure of the compressed gas supplied by the one or more second spargers to the downcomer section of the main exterior body.

28. The system of claim 1, wherein the system comprises a draft-tube internal-loop system, wherein the main exterior body comprises a cylinder with an inner tube positioned therein.

29. The system of claim 28, wherein the riser section is positioned in the inner tube.

30. The system of claim 28, wherein the riser section is positioned on either side of the inner tube.

31. The system of claim 1, wherein the system comprises a split-cylinder system, and wherein the main exterior body comprises a cylinder with a wall or a baffle positioned therein.

32. The system of claim 1, wherein the system comprises a bubble-column system, and wherein the main exterior body comprises a cylinder.

33. The system of claim 1, wherein the system is configured to be operated such that a circular liquid motion is induced by gas from the one or more first spargers and/or the one or more second spargers.

34. The system of claim 1, wherein the system includes one or more impellers to aid in mixing.
